(19) United States
(12) Patent Application Publication
(10) Pub. No.: US 2005/0075529 A1
(43) Pub. Date: Apr. 7, 2005

(54) Brachytherapy applicator chuck

(76) Inventors: Laust Pedersen, Santa Barbara, CA (US); Jerry Barber, Ventura, CA (US); Charles R. Pitman, Buellton, CA (US); Michael Collinson, Goleta, CA (US); Russel J. Redmond, Goleta, CA (US); Claude Vidal, Santa Barbara, CA (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/729,782

(22) Filed: Dec. 5, 2003

Related U.S. Application Data
(60) Provisional application No. 60/508,613, filed on Oct. 3, 2003.

(51) Int. Cl. A61M 36/00

(52) U.S. Cl. 600/7

(57) ABSTRACT

The invention provides brachytherapy applicators and brachytherapy applicator chucks configured to reduce or prevent accumulation of blood and other contaminants therein. The brachytherapy applicator chucks can be disposable.

### CROSS-REFERENCE TO RELATED APPLICATIONS

This application claims priority from U.S. Provisional Application Ser. No. 60/508,613, filed Oct. 3, 2003.

### TECHNICAL FIELD

This invention relates to devices for delivering brachytherapy seeds to an individual.

### BACKGROUND

Brachytherapy is a form of cancer treatment in which radiation sources are placed inside a patient's body to irradiate a tumor. In brachytherapy, a surgeon usually implants several radioactive seeds in or around a tumor, thus providing a radiation dose to the tumor. Careful placement of the radioactive seeds allows localized and precise irradiation of the tumor. Because the radiation dose diminishes rapidly outside the radioactive seed, the radiation dose to surrounding healthy tissues is minimized. Many forms of cancer respond to brachytherapy, including several forms of prostate cancer. Brachytherapy generally is less invasive than surgery, usually results in fewer side effects than surgery or external beam radiation, allows for a short recovery time, and reduces the impact on the patient's quality of life.

### SUMMARY

The invention features brachytherapy applicators for delivering radioactive seeds to a patient. In use, such applicators typically include a base, a chuck housing, a hollow needle that is insertable into a patient's body, a seed magazine for holding and dispensing seeds into the needle, and one or more chucks for releasably holding the needle and the seed magazine. The chuck of a brachytherapy applicator also can include a magazine retaining structure to releasably but firmly hold the seed magazine in place.

During a brachytherapy procedure, blood and other contaminants can migrate from the patient into the applicator. These blood cells and other contaminants can become lodged within the applicator in or near the magazine retaining structure. Sterilization in preparation for a future brachytherapy procedure can cause blood trapped therein to congeal, resulting in a jammed magazine retaining structure. The brachytherapy applicator chucks provided herein can be designed such that they will prevent blood cells or other particles from reaching the seed magazine retaining structure during a brachytherapy procedure. The brachytherapy applicator chucks provided herein also can be designed to allow blood cells and other contaminants to exit the applicator before reaching the magazine retaining structure.

The invention features a brachytherapy applicator chuck having a proximal end and a distal end. The chuck can define a channel extending between the proximal end and the distal end, and can be adapted to contain a needle and a septum within the channel. The septum can include an elastomeric material. The elastomeric material can contain silicone. The chuck can contain plastic, and can be disposable. The channel further can be configured to contain a radiation shield. The chuck further can define a seed magazine well and a seed magazine retention structure.

The proximal end of the chuck can be configured to hold an insert. The chuck can further contain the insert. The insert can be metal or high melting point plastic. At least a portion of the insert can have a cross-sectional diameter that is essentially the same as the cross-sectional diameter of the proximal end of the chuck.

The chuck can define one or more vents configured to permit air or contaminants to exit the chuck from the channel. The chuck can define a reservoir. The chuck can define one or more vents configured to permit air or contaminants to enter the reservoir from the channel and to exit the chuck from the reservoir.

The chuck can include a seed magazine retaining structure (e.g., a cantilever). The seed magazine retaining structure can define a protrusion configured to engage a seed magazine.

In another aspect, the invention features a brachytherapy applicator having a chuck and a chuck housing, wherein the chuck includes a proximal end and a distal end. The chuck can define a channel extending between the proximal end and the distal end, and the chuck can be adapted to contain a needle and a septum within the channel. The chuck housing can be configured to engage the chuck. The chuck further can include an insert at the proximal end. The chuck and the chuck housing can be connected by a setscrew extending from the chuck housing to contact the insert. The proximal end can define an opening extending from an exterior surface to the channel, and the setscrew can extend through the opening to contact the insert. The insert can be in direct contact with the chuck housing.

The brachytherapy applicator can further include a chuck connection device. The chuck connection device can have a proximal portion, a distal portion, and a spring lock. The chuck connection device can be configured to retain a radiation shield.

The invention also features a brachytherapy applicator chuck having a needle retention member. The chuck can have a proximal end and a distal end, and can define a channel extending between the proximal end and the distal end. The chuck can be adapted to contain a needle and a septum within the channel. The needle retention member can include a pivot structure, an actuator, and a flex beam. The flex beam can be configured to exert force against the needle. The flex beam can be configured such that it is not stressed when a needle is not present in the chuck.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a side view of a brachytherapy applicator.

FIG. 2 is a cross-sectional view of a chuck of a brachytherapy applicator.

FIG. 3 is a cross-sectional view of a chuck, a needle retention member, and a chuck connection device, where the chuck contains an insert and a septum.

FIG. 4 is a cross-sectional view of the chuck of FIG. 3, further containing a radiation shield.

FIG. 5 is an off-center, proximal end view of a chuck connected to a needle retention member and a chuck connection device, where the proximal end of the chuck is shown in cut-away view and contains an insert.

FIG. 6 is a cross-sectional view of the proximal end of a chuck, showing an alternate embodiment of an insert.

FIG. 7 is a cross-sectional view of a chuck housing, with a setscrew, a stylet, and a chuck also shown in partial cross-sectional view.

FIG. 8 is a cross-sectional view of a chuck and a chuck connection device, and a side view of a needle retention member.

FIG. 9 is a cross-sectional view of the chuck and chuck connection device of FIG. 6, where a needle and a stylet are inserted into the chuck.

FIG. 10 is a side view of a chuck and a needle retention member, with a chuck connection device shown in cut-away view.

FIG. 11 is an underside view of a chuck and a needle retention member, with a chuck connection device shown in cut-away view.

Like reference symbols in the various drawings indicate like elements.

### DETAILED DESCRIPTION

The invention provides chucks for brachytherapy applicators that can be used to deliver radioactive seeds to a patient. Brachytherapy applicators typically include a chuck, a chuck housing, and a base on which the chuck and the chuck housing can be mounted (e.g., slidably mounted). The chuck typically is configured to retain a hollow needle that is insertable into a patient's body, and a seed magazine for holding radioactive seeds and dispensing them into the needle. The chuck housing can be configured to engage the chuck, for example, and may also contain at least part of the seed magazine. The chuck and the chuck housing can be configured and connected such that seeds can be dispensed from a seed magazine into a needle in the chuck. Brachytherapy applicators also can include, for example, a stylet that is extendable through the hollow needle, chuck, chuck housing, and seed magazine.

The components of a brachytherapy applicator can be made from any suitable material, such as rigid metal or plastic materials. For example, components of a brachytherapy applicator can be made from a metal such as stainless steel, a plastic such as polysulfone or polycarbonate, and/or any other suitable material. Each component of a brachytherapy applicator can be made from a single material or from two or more materials (e.g., a metal and plastic mixture). The brachytherapy applicators provided herein can contain components such as those disclosed in, for example, U.S. Pat. Nos. 5,860,909; 5,242,373; 4,700,692; 4,461,280; and 4,402,308.

The brachytherapy applicator chucks provided herein can be disposable or reusable. Typically, brachytherapy applicators include components that are autoclavable and can be reused. As disclosed herein, however, brachytherapy applicators also can contain components that are manufactured to be relatively inexpensive and thus can be more readily disposed of by a user.

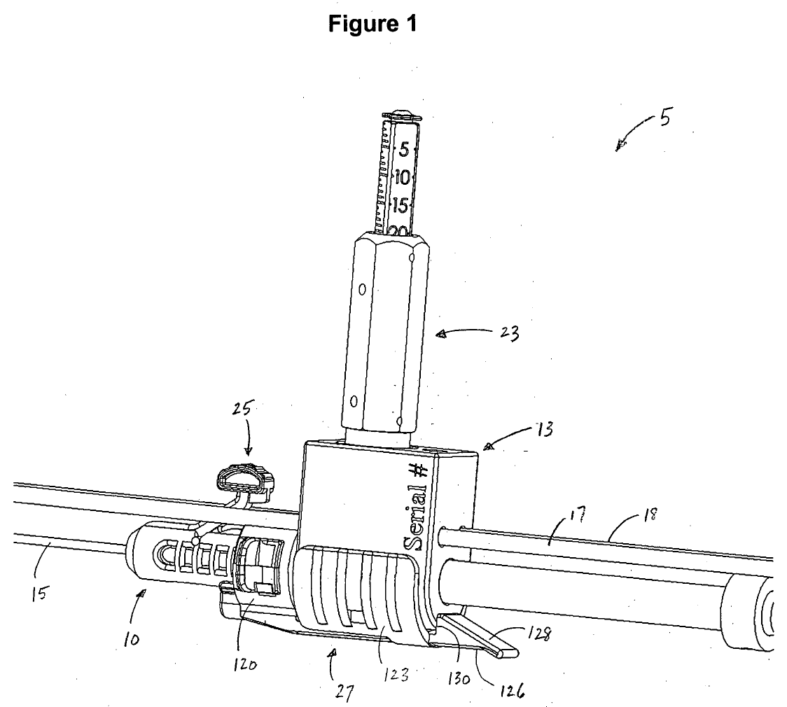

As shown in FIG. 1, brachytherapy applicator 5 can include chuck 10 and chuck housing 13, as well as needle 15 and guide rods 17 and 18. Chuck housing 13 can define a seed magazine well, which can be configured to receive and contain seed magazine 23. Brachytherapy applicator 5 also can include needle retention member 25 and chuck connection device 27. Needle retention member 25 can be configured to retain needle 15 in chuck 10, while chuck connection device 27 can be configured to hold chuck 10 and chuck housing 13 together. In some embodiments, chuck housing 13 is metal, and chuck 10 is plastic. In such embodiments, chuck housing 13 may be considered to be reusable, while chuck 10 may be more readily disposable.

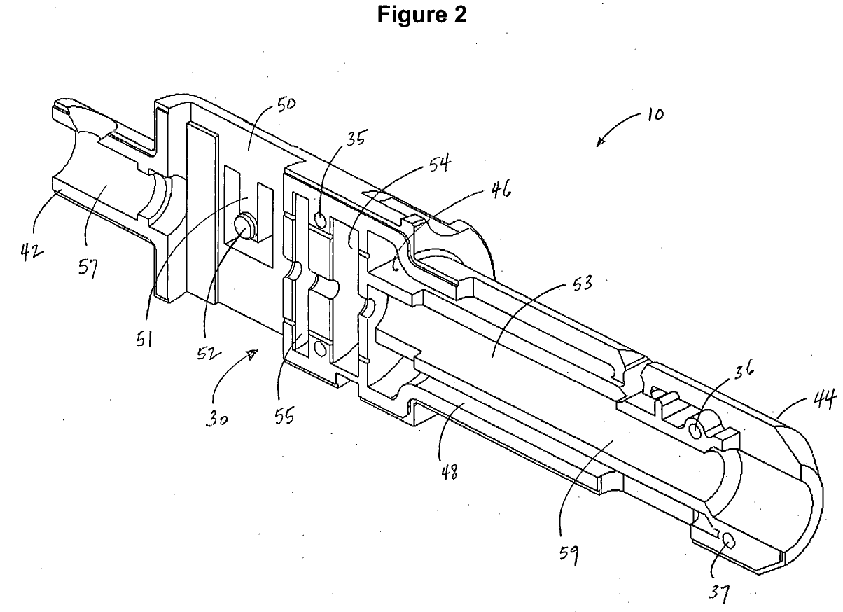

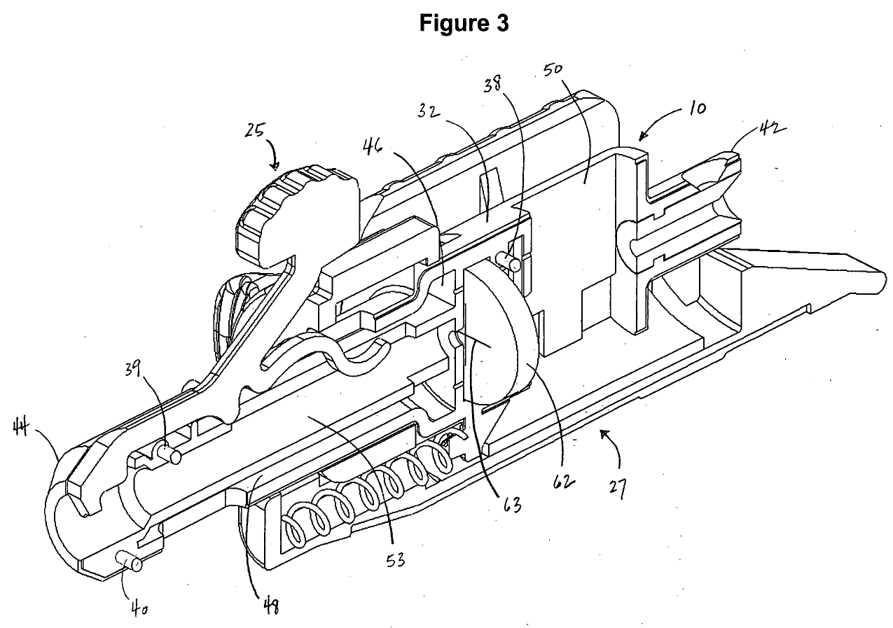

Chuck 10 can be made as a single piece or in multiple pieces. For example, as shown in FIGS. 2 and 3, respectively, chuck 10 can include a first section (e.g., first lateral section 30) and a second section (e.g., second lateral section 32). First and second lateral sections 30 and 32 can be configured to fit securely together by, for example, a pressure interference fit, to define chuck 10. In some embodiments, first and second lateral sections 30 and 32 can define pins and openings that can be configured to fit together. In these embodiments, first and second lateral sections 30 and 32 can include any number of pins and openings (e.g., one, two, three, four, five, six, seven, eight, nine, ten, or more pins and openings) configured to fit together. Furthermore, such pins and openings can be positioned anywhere on first and second lateral sections 30 and 32. For example, first lateral section 30 can include openings 35, 36, and 37, while second lateral section 32 can define pins 38, 39, and 40 that are configured to fit into openings 35, 36, and 37, respectively.

The brachytherapy applicator chucks provided herein can be configured to reduce the amount of blood that collects therein, and can prevent blood and other contaminants from reaching the seed magazine retaining device during use. Typically, the use of a brachytherapy applicator includes the slidable movement of a stylet through the chuck and the hollow needle, and into the patient to position a radioactive seed. The stylet is then retracted from the patient, through the needle, and into or through the chuck at least to a position proximal to the seed magazine. Hydrostatic pressure in the patient's body, as well as suction caused by retraction of the stylet, can cause blood and/or other contaminants to enter the chuck.

The chuck of a brachytherapy applicator can include features to reduce the likelihood of blood and other contaminants collecting within the device and/or reaching the seed magazine retaining structure. With further reference to FIG. 2, chuck 10 can define proximal end 42, distal end 44, upper reservoir 46, lower reservoir 48, and seed magazine well 50. Seed magazine well 50, which can be adapted to receive and contain at least a portion of a seed magazine (e.g., at least the distal end of a seed magazine), can be positioned in chuck 10 such that seeds can be dispensed from a seed magazine into a hollow needle. Furthermore, seed magazine well 50 can be configured to receive and reversibly retain any type of seed magazine (e.g., a Mentor or Mick seed magazine). In some embodiments, chuck 10 also can define a retaining structure to hold a seed magazine in seed magazine well 50. As shown in FIG. 2, for example, seed magazine retaining structure 51 can be configured (e.g., cantilevered) to exert force against a seed magazine to firmly but reversibly retain a seed magazine within seed magazine well 50. In one embodiment, seed magazine retaining structure 51 can define protrusion 52, which can be configured to retain a seed magazine in seed magazine well 50. For example, protrusion 52 can engage a recess in a seed magazine such that the magazine is retained within seed magazine well 50.

Chuck 10 also can define channel 53, which can extend longitudinally through the entire length of chuck 10 between proximal end 42 and distal end 44. Chuck 10 can be adapted to receive and contain a needle and a stylet within channel 53. Channel 53 can vary in width, such that chuck 10 can define, for example, septum well 54, shield well 55, insert well 57, and needle hub well 59.

Septum well 54 can be configured to contain septum 62, as shown in FIG. 3. Septum 62 can prevent blood and other contaminants from reaching a magazine during use, as described below. Septum 62 can be constructed to withstand multiple insertions of a stylet, and can be made from a self-sealing, elastomeric material such as silicone, for example. Alternatively, septum 62 can have a soft inner portion (e.g., silicone containing a hydrogel or having a lower modulus), and a less flexible, harder (e.g., plastic or silicone with a higher modulus) outer section. Such construction can enable septum 62 to essentially seal around the periphery of a stylet inserted through septum 62. Thus, when a stylet is retracted from the body of a patient and moved toward proximal end 42 of chuck 10, septum 62 can essentially wipe blood and other material from the stylet, thus preventing such contaminants from entering chuck 10 proximal to septum 62.

Septum 62 can have any shape and size. As shown in FIG. 3, for example, septum 62 can be a circular disc. Alternatively, septum 62 can be square, oval, or rectangular, for example. Septum 62 can have any thickness. For example, septum 62 can have a thickness between about 0.5 mm and about 2 mm (e.g., between about 0.7 mm and about 1.8 mm, or between about 1 mm and about 1.5 mm). Septum 62 also can define one or more openings such as slit 63, through which a stylet can be slidably movable. Slit 63 can have any size. Typically, slit 63 is about as long as the diameter of a stylet or a seed used with a brachytherapy applicator. For example, slit 63 can be at least about 0.4 millimeter (e.g., at least about 0.5 mm, 0.6 mm, 0.7 mm, 0.75 mm, 0.8 mm, 1 mm, 1.25 mm, 1.5 mm, 1.75 mm, 2 mm, 2.5 mm, or more than 2.5 mm) in length.

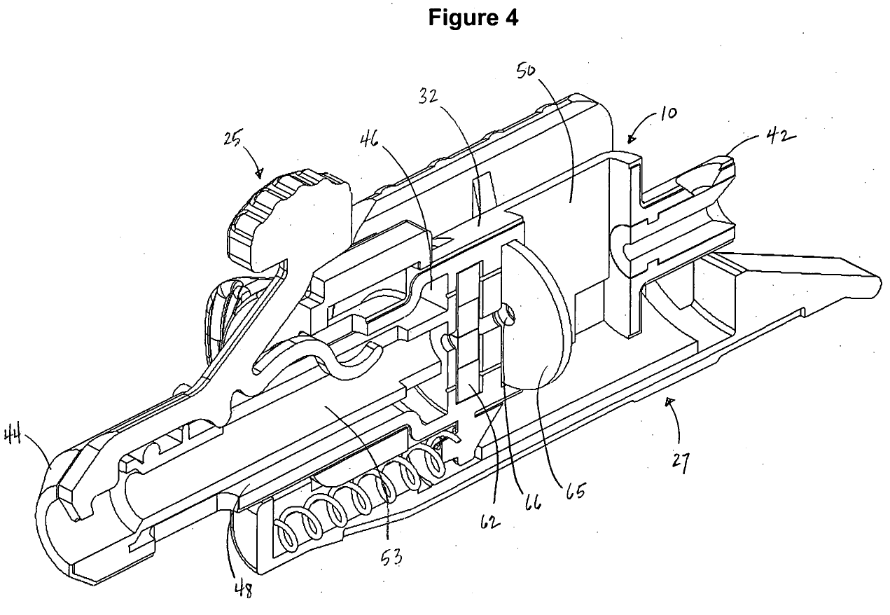

Shield well 55 can be adapted to contain inner radiation shield 65, as shown in FIG. 4. Inner radiation shield 65 can be made from a radiation shielding material (e.g., lead, stainless steel, plastic filled with particles of a metal such as lead, stainless steel, brass, or tungsten, or plastic filled with compounds of barium or bismuth). Inner radiation shield 65 can be configured to provide protection from radiation emitted by seeds in a seed magazine contained in chuck housing 13. Inner radiation shield 65 can have any shape and any dimensions. As shown in FIG. 4, for example, inner radiation shield 65 can be a circular disc. Alternatively, inner radiation shield 65 can be, without limitation, oval or polygonal (e.g., square, rectangular, or octagonal), provided that inner radiation shield 65 can be contained within shield well 55. In addition, inner radiation shield 65 can define opening 66 through which a stylet can slidably move.

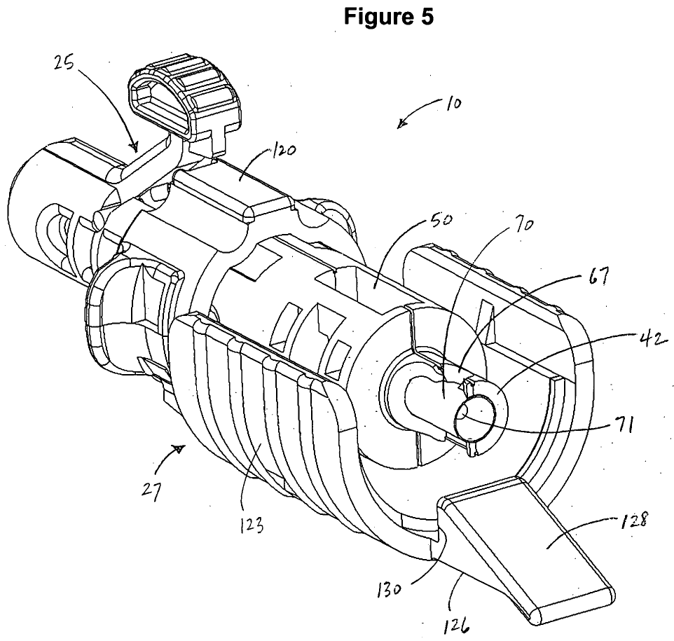

Proximal end 42 of chuck 10 can be configured to engage a chuck housing. For example, proximal end 42 can define an extension that can be inserted into an opening defined by a chuck housing. In such an embodiment, chuck 10 can be retained in a chuck housing by, for example, a setscrew that extends through the chuck housing and exerts force against proximal end 42. Alternatively, as shown FIG. 5, for example, proximal end 42 of chuck 10 can define opening 67, and channel 53 can define insert well 57, configured to hold insert 70. In this configuration, a setscrew can extend from a chuck housing and through opening 67 to contact insert 70. Insert 70 can provide a bearing surface for contacting the setscrew. Thus, a setscrew can exert force against insert 70 and hold a chuck housing and chuck 10 together.

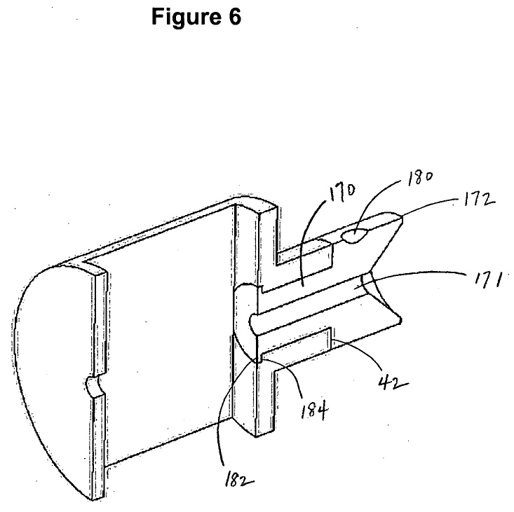

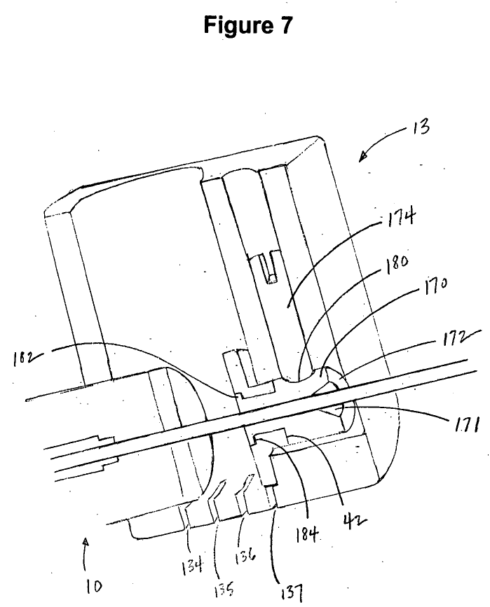

In another embodiment shown in FIGS. 6 and 7, chuck 10 can include insert 170, which can extend beyond proximal end 42 of chuck 10. In such an embodiment, insert 170 can be inserted into an opening defined by a chuck housing. In addition, proximal end 172 of insert 170 can have a cross-sectional diameter that is about the same as the cross-sectional diameter of proximal end 42 of chuck 10. Furthermore, setscrew 174 extending from chuck housing 13 can contact insert 170 without extending through chuck 10. Insert 170 can provide indentation 180 as a bearing surface for contacting setscrew 174. Insert 170 can be configured to be directly adjacent to chuck housing 13, such that force exerted on insert 170 by setscrew 174 can push insert 170 against a surface of chuck housing 13 opposite that from which the setscrew extends.

Inserts 70 and 170 can be made from any suitable material, such as metal (e.g., stainless steel) or high melting point plastic, for example. Typically, inserts 70 and 170 are constructed to withstand the force exerted by a setscrew, thus reducing the risk of cracking or breaking, or becoming deformed during autoclaving, as compared to embodiments in which a setscrew exerts force directly on an exterior surface of chuck 10.

With reference to FIGS. 6, 7, and 8, inserts 70 and 170 can define insert channels 71 and 171, which can be configured so that a stylet can slidably move therethrough. In some embodiments, insert channels 71 and 171 can be wider at their proximal ends, which can facilitate insertion of a stylet through inserts 70 and 171. Insert 70 also can define a protrusion or a cut-out (e.g., notch 72), which can mate with a cut-out or protrusion (e.g., protrusion 74) in chuck 10. Engagement of notch 72 and protrusion 74 can retain insert 70 in channel 53. Similarly, insert 171 can define a cut-out or protrusion (e.g., protrusion 182) that can engage a protrusion or a cut-out (e.g., notch 184) in chuck 10.

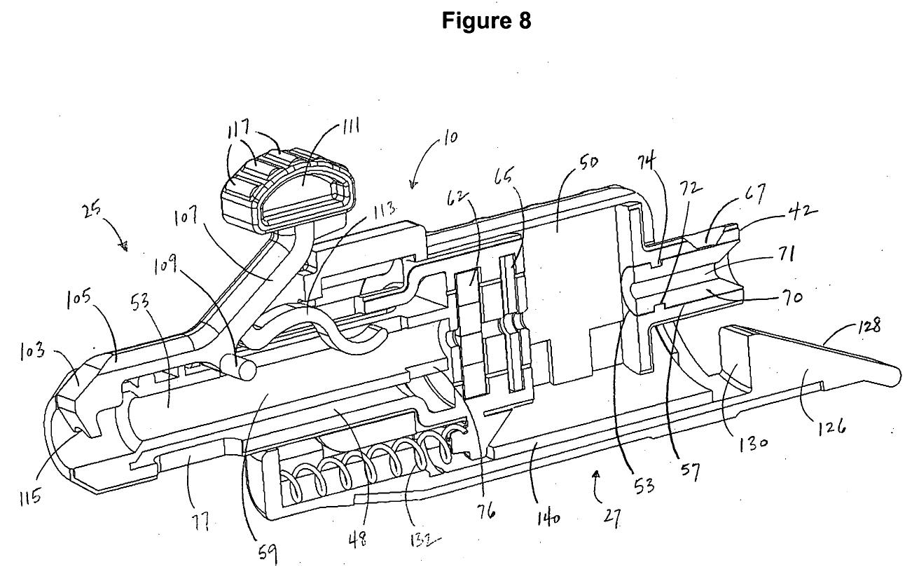

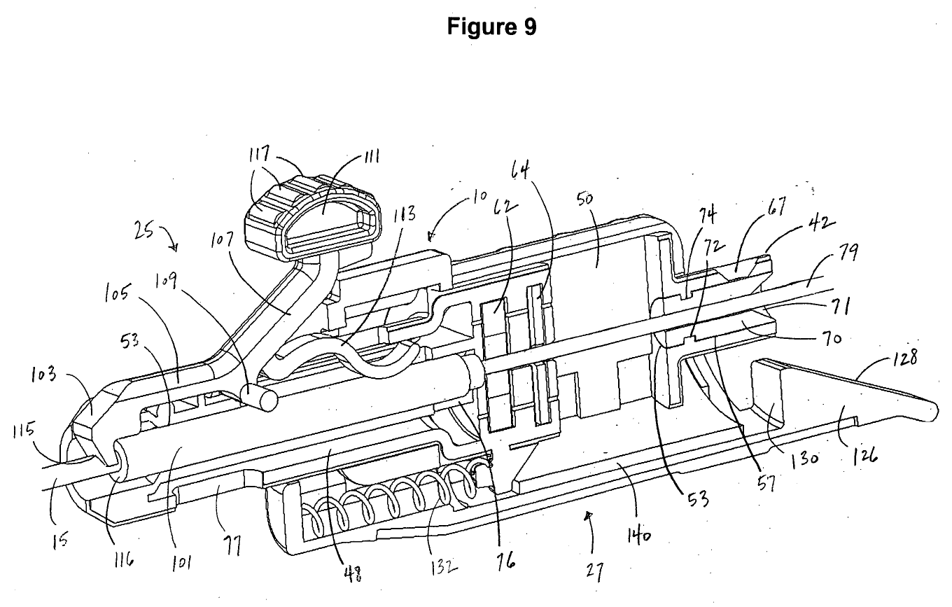

FIG. 8 provides a cross-sectional view of, inter alia, a chuck assembly including chuck 10, septum 62, inner radiation shield 65, and insert 70. FIG. 9 provides the same view, but with the addition of needle 15 and stylet 79. Needle hub well 59 of channel 53 can have any length (e.g., between about 12 mm and about 50 mm, or about 25 mm) and any diameter (e.g., between about 1.25 mm and about 5.5 mm, or about 2.7 mm) and can be configured to contain hub 101 of needle 15.

With reference to FIGS. 8 and 9, channel 53 also can define vent 76, which can extend from channel 53 to lower reservoir 48, for example. Vent 76 can be positioned distal to septum 62. When stylet 79 is withdrawn from a patient's body and pulled through needle 15 toward proximal end 42 of chuck 10, air and contaminants (e.g., blood) can enter needle 15 and channel 53. Vent 76 can be configured such that at least a portion of such air and contaminants can exit channel 53 and enter lower reservoir 48, rather than collect inside chuck 10 or inside a chuck housing to which chuck 10 is connected. Chuck 10 also can define vent 77 that extends from lower reservoir 48 to the exterior of chuck 10. Vent 77 can allow air, blood, and other contaminants to exit chuck 10.

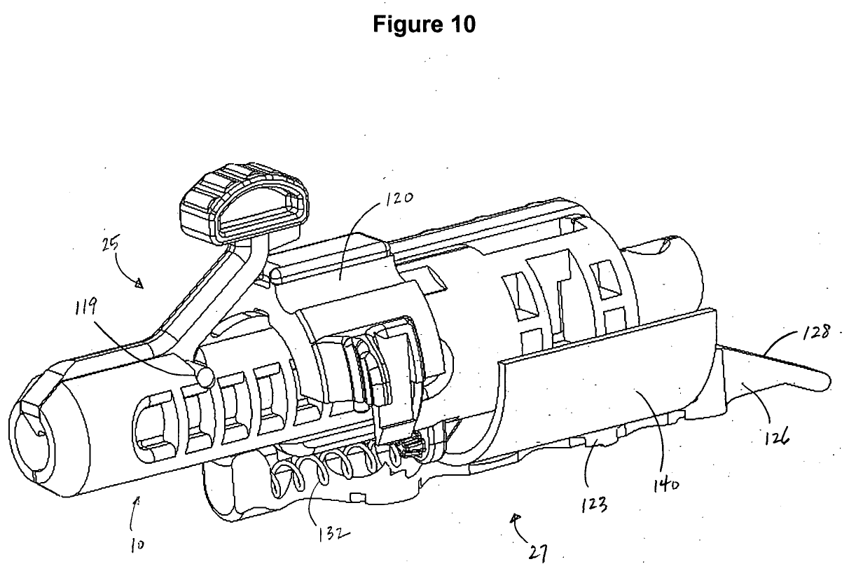

With further reference to FIGS. 8 and 9, a chuck also can include needle retention member 25. Needle retention member 25 can be configured to releasably retain needle 15 in chuck 10, by exerting force against hub 101 of needle 15. In particular, needle retention member 25 can have catch 103, arm 105, arm 107, pivot structure 109, actuator 111, and flex beam 113. Catch 103 can define contoured surface 115, which can be configured to abut against the outer surface of needle 15 (e.g., at distal end 116 of hub 101). Arms 105 and 107 can extend radially away from pivot structure 109, which can be configured to fit into a recess or opening in chuck 10. For example, chuck 10 can define recess 117 (shown in FIG. 10) to mate with pivot structure 109, such that needle retention member 25 can be retained in chuck 10 by, e.g., a snap fit. In some embodiments, lateral sections 30 and 32 of chuck 10 can define openings into which pivot structure 109 can be inserted (e.g., when chuck 10 is assembled).

Actuator 111 can be positioned at the distal end of arm 107, such that when a user exerts downward force against actuator 111, needle retention member 25 pivots about pivot structure 109, catch 103 moves up and away from channel 53, and needle 15 can be inserted into or removed from chuck 10. In some embodiments, actuator 111 can have a surface texture such as ribs 119.

Flex beam 113 can extend from arm 107 and into chuck 10, and can be configured to exert a spring action force on needle 15 (e.g., on needle hub 101) when needle 15 is present in channel 53. The force exerted against needle 15 by flex beam 113 can push catch 103 against distal end 116 of needle hub 101, thus causing needle 15 to be retained within channel 53 of chuck 10. Flex beam 113 can be configured as shown in FIGS. 8 and 9, such that its spring action is only active when needle 15 is present in chuck 10. In these embodiments, flex beam 113 is stressed when channel 53 contains a needle, and flex beam 113 is not stressed when chuck 10 does not contain a needle. Such a configuration can be useful to reduce stress-induced deformation (i.e., plastic set) during autoclaving, for example.

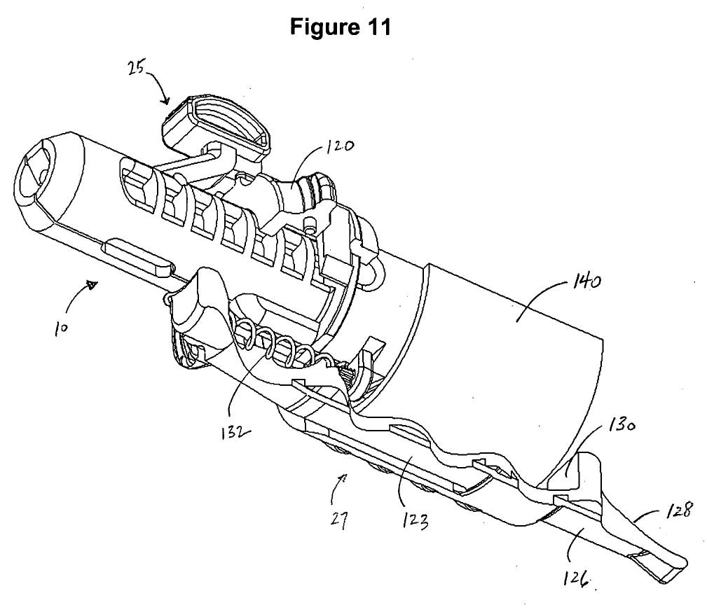

With reference to FIGS. 1, 5, and 8-11, a brachytherapy applicator also can include chuck connection device 27. Chuck connection device 27 can be configured to fit against chuck 10 and chuck housing 13, thus holding chucks 10 and 13 together. Chuck connection device 27 can be used in conjunction with a setscrew connecting the chucks 10 and 13, as disclosed above. Alternatively, chuck connection device 27 can be the only means by which chuck 10 and chuck housing 13 are held together.

Chuck connection device 27 can include distal portion 120, proximal portion 123, and spring lock 126. Distal portion 120 can be configured to encircle or otherwise contain chuck 10, while proximal portion 123 can be configured to contain chuck housing 13. Spring lock 126 can have top surface 128 and distal end 130, and can be configured such that distal end 130 fits against chuck housing 13.

Chuck connection device 27 also can be configured to contain spring 132, which can be positioned within chuck connection device 27 such that it exerts force against chuck 10 and an interior surface of chuck connection device 27. The force exerted by spring 132 can push distal end 130 of spring lock 126 against chuck housing 13, thus holding chuck housing 13 and chuck 10 together. To release chuck 10 from chuck housing 13, a user can exert force against top surface 128 of spring lock 126 to push spring lock 126 away from chuck housing 13. To assemble the chucks, a user can insert proximal end 42 of chuck 10 into chuck housing 13, push spring 132 within chuck connection device 27 against chuck 10, push proximal portion 123 against chuck housing 13, and release chuck connection device 27 such that the force exerted by spring 132 can push spring lock 126 against chuck housing 13.

Chuck connection device 27 can be configured to contain radiation shield 140, which can provide protection from, for example, radiation emitted through chuck housing 13 (e.g., via vents 134, 135, 136, and 137, shown in FIG. 7). Radiation shield 140 can be adapted to be contained within proximal portion 123, for example. Radiation shield 140 can be made from a radiation shielding material such as, for example, lead, stainless steel, plastic filled with particles of a metal such as lead, stainless steel, or tungsten, or plastic filled with compounds of barium or bismuth.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A brachytherapy applicator chuck, wherein said chuck comprises a proximal end and a distal end, said chuck defining a channel extending between said proximal end and said distal end, and wherein said chuck is adapted to contain a needle and a septum within said channel.

2. The brachytherapy applicator chuck of claim 1, wherein said septum comprises an elastomeric material.

3. The brachytherapy applicator chuck of claim 2, wherein said elastomeric material comprises silicone.

4. The brachytherapy applicator chuck of claim 1, wherein said chuck comprises plastic.

5. The brachytherapy applicator chuck of claim 1, wherein said chuck is disposable.

6. The brachytherapy applicator chuck of claim 1, wherein said channel further is configured to contain a radiation shield.

7. The brachytherapy applicator chuck of claim 1, wherein said chuck defines a seed magazine well and a seed magazine retention structure.

8. The brachytherapy applicator chuck of claim 1, wherein said proximal end is configured to hold an insert.

9. The brachytherapy applicator chuck of claim 8, further comprising said insert.

10. The brachytherapy applicator chuck of claim 9, wherein said insert is metal or high melting point plastic.

11. The brachytherapy applicator chuck of claim 9, wherein at least a portion of said insert has a cross-sectional diameter essentially the same as the cross-sectional diameter of said proximal end.

12. The brachytherapy applicator chuck of claim 1, wherein said chuck defines one or more vents, said vents being configured to permit air or contaminants to exit said channel.

13. The brachytherapy applicator chuck of claim 1, wherein said chuck defines a reservoir.

14. The brachytherapy applicator of claim 13, wherein said chuck defines one or more vents, said vents being configured to permit air or contaminants to enter said reservoir from said channel and to exit said chuck from said reservoir.

15. The brachytherapy applicator chuck of claim 1, wherein said chuck comprises a seed magazine retaining structure.

16. The brachytherapy applicator chuck of claim 15, wherein said seed magazine retaining structure is a cantilever.

17. The brachytherapy applicator chuck of claim 15, wherein said seed magazine retaining structure defines a protrusion, and wherein said protrusion is configured to engage a seed magazine.

18. A brachytherapy applicator comprising a chuck and a chuck housing, wherein said chuck comprises a proximal end and a distal end, said chuck defining a channel extending between said proximal end and said distal end, wherein said chuck is adapted to contain a needle and a septum within said channel, and wherein said chuck housing is configured to engage said chuck.

19. The brachytherapy applicator of claim 18, wherein said chuck further comprises an insert at said proximal end, and wherein said chuck and said chuck housing are connected by a setscrew extending from said chuck housing to contact said insert.

20. The brachytherapy applicator of claim 19, wherein said proximal end defines an opening extending from an exterior surface to said channel, and wherein said setscrew extends through said opening to contact said insert.

21. The brachytherapy applicator of claim 19, wherein said insert is in direct contact with said chuck housing.

22. The brachytherapy applicator of claim 18, further comprising a chuck connection device.

23. The brachytherapy applicator of claim 22, wherein said chuck connection device comprises a proximal portion, a distal portion, and a spring lock.

24. The brachytherapy applicator of claim 22, wherein said chuck connection device is configured to retain a radiation shield.

25. A brachytherapy applicator chuck comprising a needle retention member, wherein said chuck comprises a proximal end and a distal end, said chuck defining a channel extending between said proximal end and said distal end, and wherein said chuck is adapted to contain a needle and a septum within said channel.

26. The brachytherapy applicator chuck of claim 25, wherein said needle retention member comprises a pivot structure, an actuator, and a flex beam.

27. The brachytherapy applicator chuck of claim 26, wherein said flex beam is configured to exert force against said needle.

28. The brachytherapy applicator chuck of claim 26, wherein said flex beam is configured such that it is not stressed when a needle is not present in said chuck.
